(19) United States
(12) Patent Application Publication
(10) Pub. No.: US 2024/0050651 A1
(43) Pub. Date: Feb. 15, 2024

(54) MEDICAL DEVICE SYSTEM, AND DISPOSABLE CONTAINER

(71) Applicant: Roche Diabetes Care, Inc., Indianapolis, IN (US)

(72) Inventor: Harald von Campenhausen, Ladenburg (DE)

(21) Appl. No.: 18/491,782

(22) Filed: Oct. 22, 2023

Related U.S. Application Data
(63) Continuation of application No. PCT/EP2022/060395, filed on Apr. 20, 2022.

(30) Foreign Application Priority Data
Apr. 22, 2021 (EP) 21 169 885.7

(51) Int. Cl. A61M 5/142; A61J 1/18

(52) U.S. Cl. CPC A61M 5/14248; A61J 1/18; A61M 2202/0486

(57) ABSTRACT

A medical system has a body-wearable medical device such as an insulin pump, a continuous glucose monitor, or a handheld diabetes management device. The system also includes a disposable container that has a reservoir for storing a foodstuff or a drug. The disposable container has an opening that provides access to the reservoir and a tamper evident closure that seals the opening. A fastener is provided to detachably fasten the disposable container to the body-wearable medical device. The fastener may be provided in the form of a mechanical, non-adhesive medical device fastener, e.g., a bracket that at least partially embraces the body-wearable medical device or the disposable container.

### RELATED APPLICATIONS

This application is a continuation of International Application Ser. No. PCT/EP2022/060395, filed Apr. 20, 2022, which claims priority to EP 21 169 885.7, filed Apr. 22, 2021, the entire disclosures of both of which are hereby incorporated herein by reference.

### BACKGROUND

This disclosure relates to a medical device system comprising a body-wearable medical device, and a disposable container comprising a reservoir volume configured for storing a foodstuff or a drug and a tamper evident closure which seals an opening in the disposable container, wherein the opening provides access to the reservoir volume.

Body-wearable medical devices are carried by patients in order to maintain or to improve their health condition in view of a serious illness, which requires the patient to monitor certain body parameter and/or to administer a drug over a long period of time with the help of the body-wearable medical device. For instance, a patient suffering from diabetes has to regularly monitor the blood glucose level and to administer a respective dose of insulin and/or carbohydrates to keep the blood glucose level within a predetermined range.

Sometimes wearing a body-wearable medical device is not enough for a patient, as there might be exceptional situations which cannot be handled by the body-wearable medical device, but which require the patient to administer an additional amount of drug or to digest an amount of a foodstuff. Accordingly, in addition to the body-wearable medical device, the patient has to carry a so-called emergency pack containing an additional amount of drug or an amount of a foodstuff, which have to be administered by the patient in case of emergency. Not having the additional amount of drug or foodstuff at hand may result in a life-threating situation. For instance, a diabetic has to carry a certain amount of carbohydrates, usually sugar, which can be digested in case of an exceptional low blood glucose level, a so-called hypoglycemic state, in order to raise the blood glucose level on a short-notice. Patients who use medical devices generally need to carry a variety of devices and accessories in order to sustain their disease management. For example, a diabetic patient on insulin therapy may carry with him an insulin pump, a replacement infusion set, extra batteries, a glucose meter, glucose meter test strips, a lancing device, an insulin vial, and a carbohydrate foodstuff. This situation is associated with the challenge of having to check and remember which items the patient needs to pack and carry to ensure that he can manage the disease. If the diabetic patient, e.g., forgets to pack extra insulin, there is a risk that the pump's insulin reservoir runs empty and the patient cannot refill the reservoir and thus runs the risk of interrupting insulin therapy, which may in turn lead to hyperglycemia. If in another scenario, the patient forgets to carry a carbohydrate containing foodstuff such as a sugar pack or solution, in the event of a hypoglycemic situation the patient is unable to treat that condition by consuming carbohydrates so as to raise the blood level to a normal level. This, in turn, may lead to unconsciousness or even death. Thus, there is a need to increase the safety of the disease management and to alleviate the burden on the patients to remember to carry extra articles important for the disease management.

### SUMMARY

This disclosure teaches a way to reduce the burden of the patient to compose the packages to be carried when leaving home.

The disposable container contains an amount of a drug or a foodstuff, which can be administered by the patient, if needed. Accordingly, the disposable container comprises a reservoir volume (also referred herein to as “reservoir”) configured for storing a foodstuff or a drug, and having a tamper evident closure. The tamper evident closure seals an opening, which, when opened, provides access to the reservoir such that the drug or foodstuff can be withdrawn from the reservoir. The tamper evident closure is used to indicate whether or not the opening has been tampered with, i.e., if the seal provided by the tamper evident closure has been broken. Accordingly, once opened, the tamper evident closure prevents resealing the container and preferably also prevents reusing the container. It may allow the user to readily and safely determine that the disposable container has been opened and thus helps to prevent use of a potentially contaminated foodstuff or drug. After withdrawal of the drug or the foodstuff from the reservoir of the disposable container, the disposable container is disposed. The opening for withdrawing foodstuff or drug from the reservoir can be formed by one or more than one opening, such as by at least 2, at least 3, at least 4 or at least 5 openings.

The disposable container may comprise a body-wearable medical device fastener configured for detachably fastening or mounting the disposable container to the body-wearable medical device, and/or the body-wearable medical device comprises a container fastener configured for detachably fastening the body-wearable medical device to the container.

In a state in which the body-wearable medical device and the disposable container are mounted to each other, they form a unit, which can be carried by the patient. Thus, by wearing the body-wearable medical device, at the same time, the patient carries the disposable container containing foodstuff or drug to be administered in case of emergency. By being mounted to the body-wearable medical device the disposable container is less likely prone to be forgotten by the patient when packing his disease management devices and accessories because the disposable container is already mounted on the body-wearable medical device when the patient is leaving home, the office, or any other place, where the patient needs to have access to an additional amount of a drug or a foodstuff, which can be administered in case of an emergency.

The mounting of the body-wearable medical device and the disposable container is reversible, such that the disposable container can be mounted to and demounted from the body-wearable medical device. Reversible mounting is convenient for a patient so he can withdraw or administering the drug or foodstuff contained in the reservoir volume of the disposable container without having to remove the body-wearable medical device from the body. Once emptied, the disposable container can be disposed and replaced by a new disposable container, which again can be detachably mounted on the body-wearable medical device.

In accordance with this disclosure, the term disposable container refers to a container that is intended for being used one-time only. I.e., after opening the tamper-evident closure and withdrawing content out of the reservoir, the container is disposed, and should not be re-filled or re-used. Thereby, an abuse of the disposable container is less likely. In particular, once opened the contents of the reservoir can be contaminated, which may adversely affect the integrity, therapeutic effectivity and hygiene of the drug or foodstuff contents. Thus, the disposable nature of the container and the tamper evident closure sealing the opening of the reservoir increase patient's safety.

In general, the foodstuff or drug contained in the reservoir of the disposable container can be liquid, paste, powder, granules, or a mixture thereof. Accordingly, in an embodiment, the reservoir of the disposable container is configured to hold a liquid, a gel, or a pasty or powdery or granular substance, or a mixture thereof. In an embodiment, the reservoir of the disposable container contains a foodstuff comprising a carbohydrate which can be in a liquid, a gel, or a pasty or powdery or granular substance form, or a mixture thereof. In an embodiment, the foodstuff comprises a carbohydrate the consumption of which increases the blood glucose level. Preferably, the carbohydrate comprises sugar, such as fructose, glucose, or dextrose. In another embodiment, the reservoir contains a drug which can be in liquid, a gel, or a pasty or powdery or granular substance form, or a mixture thereof. The drug can be any drug that can be stored under temperature conditions in the disposable container of, e.g., in a range between 10 degrees to 30 degrees, preferably at room temperature, without significantly adversely affecting the therapeutic efficacy of the stored drug for an interval of time of up to 1 hour, up to 2 hours, up to 5 hours, up to 7 hours, up to 10 hours, up to 1 day, up to 2 days, up to 3 days, up to 5 days, up to 7 days.

In an embodiment, the drug is a drug for the treatment of a disease selected from cancer, a chronic disease such as diabetes or asthma, a cardiovascular disease, a metabolic disease, an autoimmune disease or an infectious disease. Preferably the drug is used for treating diabetes, preferably the drug is an insulin. In an embodiment the insulin includes a rapid-acting, a short-acting, an intermediate-acting, a long-acting or a pre-mixed insulin, In an embodiment the insulin is selected from Lispro (Humalog), Aspart (Novolog), Glulisine (Apidra), Regular (R) or Novolin, Velosulin, NPH, Insulin glargine (Basaglar, Lantus, Toujeo), Insulin detemir (Levemir), Insulin degludec (Tresiba), Humulin, and Novolog.

According to an embodiment of the medical device system, the body-wearable medical device fastener is or comprises a mechanical, non-adhesive medical device fastener and/or the disposable container fastener is or comprises a mechanical, non adhesive disposable container fastener. A mechanical, non-adhesive medical device fastener or a mechanical, non-adhesive disposable container fastener can be removed free of residues. In particular, upon removal of a mechanical, non-adhesive fastener there is no sticky residue left on the disposable container or the body-wearable medical device, respectively, which otherwise may cause discomfort to a user. In an embodiment, the term non-adhesive fastener is meant to specify that the fastener is free of a substance (such as a glue) for sticking the disposable container and the body-wearable medical device together.

According to an embodiment of the medical device system, the body-wearable medical device fastener is or comprises a medical device bracket configured for at least partially embracing the body-wearable medical device and/or the disposable container fastener is or comprises a disposable container bracket configured for at least partially embracing the container. In the meaning of this disclosure, a medical device bracket configured for at least partially embracing the body-wearable medical device or disposable container bracket for at least partially embracing the disposable container comprises a first portion and second portion, which are connected to each by a bridging portion such that during use, i.e., when embracing the body-wearable medical device or disposable container, respectively, the first portion and second portion of the respective bracket are arranged on opposing sides of the body-wearable medical device or disposable container, respectively. The first portion, second portion, and bridging portion can be configured to provide a clamping force for detachable fastening the body-wearable medical device to the disposable container, or vice versa. The first portion and second portion each can be a part of respective pairs of correspondingly configured fastener pairs as for instance described below.

According to an embodiment of the medical device system, the disposable container comprises a circumferential edge, which, in a state of the disposable container fastened to the body-wearable medical device, circumferentially contacts the body-wearable medical device. The circumferential edge of the container contacting the body-wearable medical device stabilize the connection between the body-wearable medical device and the container. Furthermore, the contacting circumferential edge prevent foreign matter entering between the body-medical device and the container. In a further modified embodiment, the circumferential edge is configured for clamping the disposable container to the body-wearable medical device thereby being configured as the body-wearable medical device fastener or at least part of the body-wearable medical device fastener.

According to an embodiment of the medical device system, the disposable container fastener and the body-wearable medical device fastener interoperate to detachably fasten the disposable container to the body-wearable medical device. The disposable container fastener and the body-wearable medical device fastener form a pair of engaging mechanical elements. In a state, the disposable container and the body-wearable medical device are detached from each other, the disposable container fastener remains at the body-wearable medical device, while the body-wearable medical device fastener remains at the disposable container. According to a further modified embodiment, the interoperating disposable container fastener and body-wearable medical device fastener are configured to allow fastening the disposable container to the body-wearable medical device in a single pre-determined position defined by the respective arrangement of the interoperating disposable container fastener and body-wearable medical device fastener.

According to an embodiment of the medical device system, the disposable container fastener and the body-wearable medical device fastener form a pair of hook-and-loop fastener or a pair of hook-and-eye fastener or a latch fastener or clip fastener.

According to an embodiment of the medical device system, the reservoir volume is up to 20 cm³, alternatively the reservoir volume is up to 15 cm³, further alternatively the reservoir volume is equal to 13 cm³. In any case, the reservoir volume is larger 0 cm³, so that the reservoir volume can contain a foodstuff or a drug.

In order to ease withdrawal of a foodstuff or a drug contained in the reservoir of the disposable container via the opening, according to an embodiment of the medical device system, the disposable container comprises a rip-open venting closure sealing a venting opening of the reservoir. This additional venting opening allows venting the reservoir of the disposable once the rip-open venting closure is opened such that withdrawal of foodstuff or drug via the opening of the disposable container is not hindered by negative pressure or a reverse flow of air trying to enter the reservoir through the opening for withdrawing foodstuff or drug. Such venting opening is particularly useful if the foodstuff or liquid contained in the reservoir is in liquid or gel form. A rip-open closure can be opened without using any tools. The rip-open venting closure that seals the venting opening and the tamper-evident closure sealing the opening for withdrawing foodstuff or drug from the reservoir volume can be a combined in one closure. I.e., the venting closure and the tamper-evident closure can be removed together so that the venting opening and the opening for withdrawing foodstuff or drug from the reservoir volume are opened together.

In an embodiment of the medical device system the disposable container, except for the body-wearable medical device fastener, is an envelope or pouch, preferably the envelope or pouch is pre-filled with a foodstuff or a drug. In the meaning of this disclosure, an envelope or pouch is a flexible container. The wall thickness of the envelope or pouch can be equal to or smaller than 1 mm. The body-wearable medical device fastener can be but does not need to be flexible like the remainder of the disposable container. In an embodiment, the disposable container is entirely or at least partially made of a plastic. For instance, the plastic is or comprises polycarbonate, polyethylene, polypropylene, or a mixture thereof.

This disclosure further relates to a disposable container comprising a reservoir volume configured for storing a foodstuff or a drug, and a tamper evident closure which seals an opening in the disposable container, the opening provides access to the reservoir volume, wherein the disposable container comprises a body-wearable medical device fastener configured for detachable fastening the disposable container to a body-wearable medical device. The disposable container can be configured as described above in the context of the medical device system. By way of example only, the body-wearable medical device can be configured as described in the context of the medical device system above, or as described further below.

According to an embodiment of the disposable container, the body-wearable medical device fastener is or comprises a mechanical, non-adhesive medical device fastener.

According to an embodiment of the disposable container, the body-wearable medical device fastener is or comprises a medical device bracket configured for at least partially embracing the body-wearable medical device.

According to an embodiment of the disposable container, the disposable container comprises a circumferential edge, which, in a state of the disposable container fastened to the body-wearable medical device, circumferentially contacts the body-wearable medical device.

According to an embodiment of the disposable container, the body-wearable medical device fastener is configured to interoperate with a disposable container fastener of the body-wearable medical device to detachably fasten the disposable container to the body-wearable medical device.

According to an embodiment of the disposable container, the body-wearable medical device fastener is a portion of a pair of hook-and-loop fastener or a portion of a pair of hook-and-eye fastener or a portion of a latch fastener or a portion of a clip fastener.

According to an embodiment of the disposable container, the reservoir volume is up to 20 cm³, alternatively the reservoir volume is up to 15 cm³, further alternatively the reservoir volume is equal to 13 cm³. In any case, the reservoir volume of the disposable container is larger than 0 cm³ so that the reservoir volume can contain a drug or a foodstuff.

According to an embodiment of the disposable container, the container comprises a rip-open venting closure sealing a venting opening of the reservoir volume.

According to an embodiment of the disposable container, the disposable container is an envelope or pouch, preferably the envelope or pouch is pre-filled with a foodstuff or a drug as outlined above.

This disclosure further relates to a body-wearable medical device, including a disposable container fastener configured for detachably fastening a disposable container to the body-wearable medical-device. The body-wearable medical device can be configured as described above in the context of the medical device system. By way of example only, the disposable container can be configured as described in the context of the medical device system above.

According to an embodiment of the body-wearable medical device is a body wearable drug infusion pump, in a preferred embodiment a body wearable drug infusion patch pump, including an insulin pump. Alternatively, the body-wearable medical device may be a body wearable in vitro or in vivo analyte sensor including a continuous glucose measurement device. Other body wearable medical devices include a body wearable handheld remote control of a controlled analyte sensor device or drug infusion device, such as a smartphone a wearable such as a watch or a proprietary remote control such of the above such as a diabetes management device.

According to an embodiment of the body-wearable medical device, the disposable container fastener is or comprises a mechanical, non-adhesive disposable container fastener.

According to an embodiment of the body-wearable medical device, the disposable container fastener is or comprises a disposable container bracket configured for at least partially embracing a container.

According to an embodiment of the body-wearable medical device, the disposable container fastener comprises a circumferential edge, which, in a state of a disposable container fastened to the body-wearable medical device, circumferentially contacts the disposable container.

According to an embodiment of the body-wearable medical device, the disposable container fastener is configured to interoperate with body-wearable device fastener of a disposable container to detachably fasten the disposable container to the body-wearable medical device.

According to an embodiment of the body-wearable medical device, the disposable container fastener forms a portion of a pair of hook-and-loop fastener or a portion of a pair of hook-and-eye fastener or a portion of a latch fastener or a portion of a clip fastener.

Insofar as in the foregoing as well as in the following detailed description of embodiments and claims, features of the disposable container described in the context of a body-wearable medical device system are applicable to the disposable container on its own, i.e., irrespective of the presence of a body-wearable medical device. Likewise, insofar as in the foregoing as well as in the following detailed description of embodiments and claims, features of the body-wearable medical device described in the context of a body-wearable medical device system are applicable to the body-wearable medical device on its own, i.e., irrespective of the presence of a disposable container.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows a schematic view of a medical device system according to an embodiment of this disclosure; and

FIG. 2 shows a schematic view of a medical device system according to another embodiment of this disclosure.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

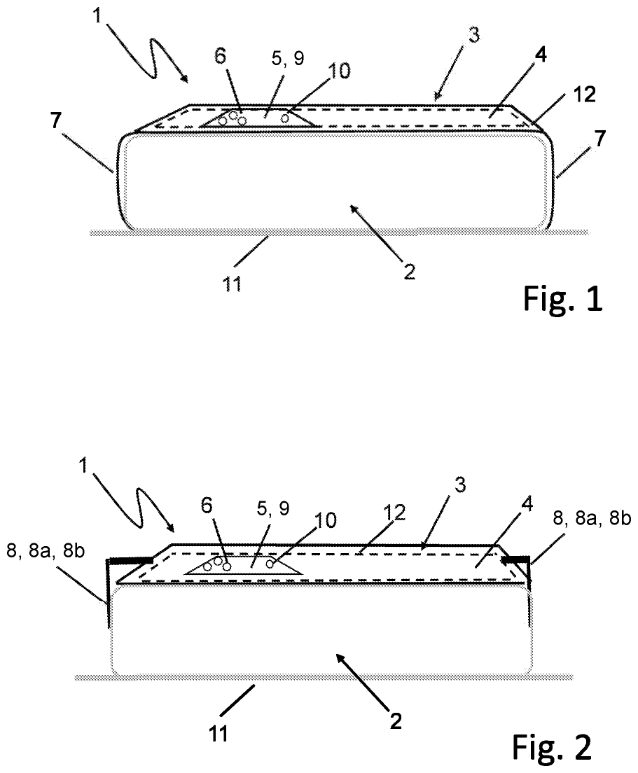

FIG. 1 shows a schematic view of a medical device system 1 according to an embodiment of this disclosure. The medical device system 1 comprises a body-wearable medical device 2 and disposable container 3. The schematic view shows the disposable container 3 in a state detachably fastened to the body-wearable medical device 2. The body-wearable medical device 2 is visualized in a state attached to a skin 11 of the patient. The body-wearable medical device 2 is a drug infusion pump, such as an insulin pump, or continuous glucose measurement device.

The disposable container 3 comprises a volume reservoir 4 containing a foodstuff or a drug. An opening 6 of the disposable container 3 provides access to the volume reservoir 4. When opened the foodstuff or the drug can be withdrawn from the volume reservoir 4 through the opening 6. For a smooth withdrawal of foodstuff or drug particularly in liquid or gel state, the disposable container 3 further comprises a venting opening 10. The venting opening 10 allows a flow of air flowing into the volume reservoir 4 when the foodstuff or the drug is withdrawn out of the volume reservoir 4 via the opening 6. The venting opening 10 is optional.

Further optional, in the shown embodiment, the opening 6 for withdrawing a foodstuff or a drug out of the volume reservoir 4 is formed by multiple holes.

Initially the opening 6 and the venting opening 10 are sealed by a single, combined tamper-evident closure comprising the rip-open venting closure 9 and the tamper-evident closure 5. Accordingly, the rip-open venting closure 9 sealing the venting opening 10, and the tamper-evident closure 5 form a single element. The tamper-evident closure 5 seals the opening 6 for withdrawing foodstuff or drug out of the volume reservoir 4.

The disposable container 3 comprises a body-wearable medical device fastener 7. The body-wearable medical device fastener 7 is configured to detachably fasten the disposable container 3 to the body-wearable medical device fastener 7. In the present embodiment the body-wearable medical device fastener 7 is a mechanical, non-adhesive medical device fastener 7a forming a medical device bracket 7b. In a state in which the disposable container 3 is fastened to the body-wearable medical device 2, the medical device bracket 7b at least partially embraces the body-wearable medical device 2.

Furthermore, the disposable container 3 comprises a circumferential edge 12, which in a state the disposable container 3 is detachably fastened to the body-wearable medical device 2 circumferentially contacts the body-wearable medical device 2.

FIG. 2 shows another embodiment of a medical device system 1 according to this disclosure. The embodiment according to FIG. 2 differs from the embodiment shown in FIG. 1 by having disposable container fastener 8 instead of having body-wearable medical device fastener 7. The disposable container fastener 8 is configured to detachable fasten the disposable container 3 to the body-wearable medical device 2. The disposable container fastener 8 is a mechanical, non-adhesive disposable container fastener 8a in the form of a disposable container bracket 8b, which at least partially embraces the disposable container 3, when being detachable mounted to the body-wearable medical device 2.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

1 medical device system
2 body-wearable medical device
3 disposable container
4 reservoir volume
5 tamper-evident closure
6 opening
7 body-wearable medical device fastener
7a mechanical, non-adhesive medical device fastener
7b medical device bracket
8 disposable container fastener
8a mechanical, non-adhesive disposable container fastener
8b disposable container bracket
9 rip-open venting closure
10 venting opening
11 Body skin
12 circumferential edge of the disposable container 3

What is claimed is:

1. A medical system, comprising:
a body-wearable medical device selected from the group consisting of an insulin pump, a continuous glucose monitor, and a handheld diabetes management device;
a disposable container having a reservoir configured for storing a foodstuff or a drug, the disposable container having an opening that provides access to the reservoir and a tamper evident closure that seals the opening; and
a fastener configured to detachably fasten the disposable container to the body-wearable medical device.

2. The medical system according to claim 1, wherein the fastener comprises a mechanical, non-adhesive medical device fastener.

3. The medical system according to claim 1, wherein the fastener comprises a medical device bracket configured for at least partially embracing the body-wearable medical device and/or for at least partially embracing the disposable container.

4. The medical system according to claim 1, wherein the disposable container comprises a circumferential edge, which, when the disposable container is fastened to the body-wearable medical device, circumferentially contacts the body-wearable medical device.

5. The medical system according to claim 1, wherein the fastener is configured to interoperate to detachably fasten the disposable container to the body-wearable medical device.

6. The medical system according to claim 1, wherein the fastener comprises a hook-and-loop fastener, a hook-and-eye fastener, a latch fastener or clip fastener.

7. The medical system according to claim 1, wherein the reservoir has a volume selected from the group consisting of up to 20 cm³, up to 15 cm³, and equal to 13 cm³.

8. The medical system according to claim 1, wherein the disposable container comprises a rip-open venting closure sealing a venting opening of the reservoir volume.

9. A disposable container, comprising:
a reservoir configured for storing a foodstuff or a drug;
an opening that provides access to the reservoir and a tamper evident closure that seals the opening;
a body-wearable medical device fastener configured for detachably fastening the disposable container to a body-wearable medical device, wherein the body-wearable medical device is an insulin pump, a continuous glucose monitor or a handheld diabetes management device.

10. The disposable container according to claim 9, wherein the body-wearable medical device fastener comprises a non-adhesive medical device fastener.

11. The disposable container according to claim 9, wherein the body-wearable medical device fastener comprises a medical device bracket configured for at least partially embracing the body-wearable medical device.

12. The disposable container according to claim 9, wherein the disposable container comprises a circumferential edge, which, when the disposable container is fastened to the body-wearable medical device, circumferentially contacts the body-wearable medical device.

13. A body-wearable medical device comprising a disposable container fastener configured for detachably fastening a disposable container to the body-wearable medical-device, wherein the body-wearable medical device is an insulin pump, a continuous glucose monitor or a handheld diabetes management device.

14. The body-wearable medical device according to claim 13, wherein the disposable container fastener comprises a mechanical, non-adhesive disposable container fastener.
